(19) United States
(12) Patent Application Publication
(10) Pub. No.: US 2020/0405719 A1
(43) Pub. Date: Dec. 31, 2020

(54) CANCER TREATMENT USING COMBINATION OF NEUTROPHIL MODULATOR WITH MODULATOR OF IMMUNE CHECKPOINT

(71) Applicant: APOLLOMICS INC., Foster City, CA (US)

(72) Inventors: Sanjeev REDKA, Foster City, CA (US); Mammatha REDDY, Foster City, CA (US)

(21) Appl. No.: 16/970,384

(22) PCT Filed: Feb. 17, 2019
(86) PCT No.: PCT/US2019/018377

§ 371 Date: Aug. 17, 2020

Related U.S. Application Data
(60) Provisional application No. 62/631,771, filed on Feb. 17, 2018.
(60) Provisional application No. 62/757,729, filed on Nov. 8, 2018.

(51) Int. Cl. A61K 31/5025; A61P 35/00; C07K 16/28

(52) U.S. Cl. CPC A61K 31/5025; C07K 16/2827; C07K 16/2818; A61P 35/00

(57) ABSTRACT

The present disclosure provides methods of treating a cancer in a subject. The method includes a step of measuring a base level of a biomarker selected from a group consisting of hepatocyte growth factor, absolute neutrophil count, c-Met+ neutrophils and neutrophil to lymphocyte ratio (NLR) in the subject. The method also includes the steps of determining that the base level of said biomarker is equal or more than a threshold value or determining the change in the said biomarker upon administration of an immune checkpoint modulator is equal or more than a threshold value; and administering to the subject a combination of c-Met inhibitor and a modulator of an immune checkpoint.

### CROSS-REFERENCE TO RELATED APPLICATIONS

This application claims priority to U.S. provisional patent application Nos. 62/631,771, filed Feb. 17, 2018, and 62/757,729, filed Nov. 8, 2018, the disclosure of which is incorporated herein by reference.

### FIELD OF THE INVENTION

The present invention generally relates to cancer treatment. In particular, the present invention relates to methods for treating a cancer using combination of a neutrophil modulator with a modulator of immune checkpoint.

### BACKGROUND

Cancer immunotherapy that modulates a patient's own immune system to fight the tumor highlights the significance of the mechanisms that cancer cells evolve to shun immune surveillance, e.g., by promoting immune tolerance to tumor antigens expressed by cancer-associated genetic alteration. Several immune checkpoint inhibitors, represented by monoclonal antibodies against PD-1, PD-L1 or CTLA4, have yielded remarkable and durable responses for some patients with an increasingly broad array of cancer types. However, current immunotherapies as single agents, such as PD-1 or PD-L1 blockade, only exhibit limited response in cancer patients (see, e.g., Padmanee Sharma and James P. Allison, “Immune Checkpoint Targeting in Cancer Therapy: Toward Combination Strategies with Curative Potential” Cell (2015) 161: 205-214).

To extend the application of cancer immunotherapies, combination therapies that modulate the activity of immune checkpoint pathways have been explored. For example, combination of c-Met inhibitors with antibodies of PD-1 has been tested (see, e.g., WO 2017/106810; Glodde et al., Immunity (2017) 47:789-802). However, the responsiveness to the combination treatment of c-Met inhibitors and anti-PD-1 antibodies are context dependent (Glodde et al., Immunity (2017) 47:789-802). Therefore, there is a continuing need to develop new methods to increase the responsiveness of combinational immunotherapies for treating cancer.

### SUMMARY

In one aspect, the present disclosure provides a method of treating a subject having a cancer. In one embodiment, the method comprises: measuring a base level of a biomarker selected from a group consisting of hepatocyte growth factor, absolute neutrophil count, c-Met+ neutrophils and neutrophil to lymphocyte ratio (NLR) in a sample from the subject; determining that the base level of said biomarker is equal or more than a threshold value; and administering to the subject a combination of a therapeutically effective amount of a neutrophil modulator and a modulator of an immune checkpoint.

In another embodiment, the method comprises: measuring a first level of a biomarker selected from a group consisting of hepatocyte growth factor, absolute neutrophil count, c-Met+ neutrophils and NLR in the subject; administering to the subject a modulator of an immune checkpoint for a time period; measuring a second level of the biomarker in the subject; determining that a difference between the second level of the biomarker and the first level of biomarker is equal or more than a critical value; and administering to the subject a combination of a therapeutically effective amount of a neutrophil modulator and a modulator of an immune checkpoint.

Yet in another embodiment, the method of the present disclosure administering to the subject a combination of a therapeutically effective amount of a c-Met inhibitor and an anti-PD-1 antibody or an anti-PD-L1 antibody.

### BRIEF DESCRIPTION OF DRAWING

FIGS. 1A-1C illustrate the synergistic effect of a combination of c-Met inhibitor and an anti-PD-1 antibody in MC-38 syngeneic colon cancer model. FIG. 1A illustrates the design of the experiments. FIG. 1B illustrates that the combination of c-Met inhibitor (APL-101) and anti-PD-1 antibody synergistically inhibited the tumor growth. FIG. 1C illustrates that the treatment of c-Met inhibitor and anti-PD-1 antibody, alone or in combination, did not affect the body weight of the mice being treated.

FIGS. 2A-2C illustrate the synergistic effect of a combination of c-Met inhibitor and an anti-PD-1 antibody in H-22 syngeneic hepatocellular carcinoma model. FIG. 2A illustrates the design of the experiments. FIG. 2B illustrates that the combination of c-Met inhibitor (APL-101) and anti-PD-1 antibody synergistically inhibited the tumor growth. FIG. 2C illustrates that the treatment of c-Met inhibitor and anti-PD-1 antibody, alone or in combination, did not affect the body weight of the mice being treated.

FIGS. 3A-3C illustrate the synergistic effect of a combination of c-Met inhibitor and an anti-PD-1 antibody in RENCA syngeneic renal cell carcinoma model. FIG. 3A illustrates the design of the experiments. FIG. 3B illustrates that the combination of c-Met inhibitor (APL-101) and anti-PD-1 antibody synergistically inhibited the tumor growth. FIG. 3C illustrates that the treatment of c-Met inhibitor and anti-PD-1 antibody, alone or in combination, did not affect the body weight of the mice being treated.

FIGS. 4A-4C illustrate that a combination of c-Met inhibitor and an anti-PD-1 antibody deceased the neutrophil percentage in tumor microenvironment. FIG. 4A illustrates that a treatment of anti-PD-1 antibody increased c-Met positive neutrophils in an IHC analysis. FIG. 4B illustrates that a combination of a c-Met inhibitor and an anti-PD-1 antibody decreased neutrophil percentage in tumor microenvironment. FIG. 4C illustrates that a treatment of anti-PD-1 antibody increased c-Met positive neutrophils in peripheral circulation, and a combination of a c-Met inhibitor and an anti-PD-1 antibody decreased the neutrophil percentage in peripheral circulation.

FIG. 5 is a schematic of a Phase 1 study of combination immunotherapy anti-PD1 with c-Met inhibitor.

FIG. 6 is a schematic of a Phase 2 study of combination immunotherapy anti-PD1 with c-Met inhibitor.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, the term “administering” means providing a pharmaceutical agent or composition to a subject, and includes, but is not limited to, administering by a medical professional and self-administering.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)2, Fab, Fv, and rIgG). See also, e.g., Pierce Catalog and Handbook, 1994-1995 (Pierce Chemical Co., Rockford, Ill.); Kuby, J., Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

As used herein, an “anti-angiogenesis agent” means a substance that reduces or inhibits the growth of new blood vessels, such as, e.g., an inhibitor of vascular endothelial growth factor (VEGF) and an inhibitor of endothelial cell migration. Anti-angiogenesis agents include without limitation 2-methoxyestradiol, angiostatin, bevacizumab, cartilage-derived angiogenesis inhibitory factor, endostatin, IFN-α, IL-12, itraconazole, linomide, platelet factor-4, prolactin, SU5416, suramin, tasquinimod, tecogalan, tetrathiomolybdate, thalidomide, thrombospondin, thrombospondin, TNP-470, ziv-aflibercept, pharmaceutically acceptable salts thereof, prodrugs, and combinations thereof.

As used herein, the term “cancer” refers to any diseases involving an abnormal cell growth and includes all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, breast cancer, Burkitt's lymphoma, cerebellar astrocytoma, cerebral astrocytoma/malignant glioma, cervical cancer, chronic lymphocytic leukemia, chronic myelogenous leukemia, colon cancer, emphysema, endometrial cancer, ependymoma, esophageal cancer, Ewing family of tumors, Ewing's sarcoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, medulloblastoma, melanoma, neuroblastoma, non-Hodgkin lymphoma, ovarian cancer, pancreatic cancer, pharyngeal cancer, prostate cancer, rectal cancer, renal cell carcinoma (kidney cancer), retinoblastoma, skin cancer, stomach cancer, supratentorial primitive neuroectodermal tumors, testicular cancer, throat cancer, thyroid cancer, vaginal cancer, visual pathway and hypothalamic glioma.

Cytotoxic agents according to the present invention include DNA damaging agents, antimetabolites, anti-microtubule agents, antibiotic agents, etc. DNA damaging agents include alkylating agents, platinum-based agents, intercalating agents, and inhibitors of DNA replication. Non-limiting examples of DNA alkylating agents include cyclophosphamide, mechlorethamine, uramustine, melphalan, chlorambucil, ifosfamide, carmustine, lomustine, streptozocin, busulfan, temozolomide, pharmaceutically acceptable salts thereof, prodrugs, and combinations thereof. Non-limiting examples of platinum-based agents include cisplatin, carboplatin, oxaliplatin, nedaplatin, satraplatin, triplatin tetranitrate, pharmaceutically acceptable salts thereof, prodrugs, and combinations thereof. Non-limiting examples of intercalating agents include doxorubicin, daunorubicin, idarubicin, mitoxantrone, pharmaceutically acceptable salts thereof, prodrugs, and combinations thereof. Non-limiting examples of inhibitors of DNA replication include irinotecan, topotecan, amsacrine, etoposide, etoposide phosphate, teniposide, pharmaceutically acceptable salts thereof, prodrugs, and combinations thereof. Antimetabolites include folate antagonists such as methotrexate and premetrexed, purine antagonists such as 6-mercaptopurine, dacarbazine, and fludarabine, and pyrimidine antagonists such as 5-fluorouracil, arabinosylcytosine, capecitabine, gemcitabine, decitabine, pharmaceutically acceptable salts thereof, prodrugs, and combinations thereof. Anti-microtubule agents include without limitation vinca alkaloids, paclitaxel (Taxol®), docetaxel (Taxotere®), and ixabepilone (Ixempra®). Antibiotic agents include without limitation actinomycin, anthracyclines, valrubicin, epirubicin, bleomycin, plicamycin, mitomycin, pharmaceutically acceptable salts thereof, prodrugs, and combinations thereof.

As used herein, the term “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

In the present invention, the term “immunomodulator” means a substance that alters the immune response by augmenting or reducing the ability of the immune system to produce antibodies or sensitize cells that recognize and react with the antigen that initiated their production. Immunomodulators may be recombinant, synthetic, or natural preparations and include cytokines, corticosteroids, cytotoxic agents, thymosin, and immunoglobulins. Some immunomodulators are naturally present in the body, and certain of these are available in pharmacologic preparations. In certain embodiments, immunomodulators are modulators of an immune checkpoint. Examples of immunomodulators include, but are not limited to, granulocyte colony-stimulating factor (G-CSF), interferons, imiquimod and cellular membrane fractions from bacteria, IL-2, IL-7, IL-12, CCL3, CCL26, CXCL7, and synthetic cytosine phosphate-guanosine (CpG).

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

“Pharmaceutically-acceptable salts” refers to the relatively non-toxic, inorganic and organic acid addition salts of compounds.

As used herein, the term “photoactive therapeutic agent” means compounds and compositions that become active upon exposure to light. Certain examples of photoactive therapeutic agents are disclosed, e.g., in U.S. Patent Application Publication Serial No. 2011/015223.

As used herein, the term “radiosensitizing agent” means a compound that makes tumor cells more sensitive to radiation therapy. Examples of radiosensitizing agents include misonidazole, metronidazole, tirapazamine, and trans sodium crocetinate.

The terms “responsive,” “clinical response,” “positive clinical response,” and the like, as used in the context of a patient's response to a cancer therapy, are used interchangeably and refer to a favorable patient response to a treatment as opposed to unfavorable responses, i.e. adverse events. In a patient, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment. In a population the clinical benefit of a drug, i.e., its efficacy can be evaluated on the basis of one or more endpoints. For example, analysis of overall response rate (ORR) classifies as responders those patients who experience CR or PR after treatment with drug. Analysis of disease control (DC) classifies as responders those patients who experience CR, PR or SD after treatment with drug. A positive clinical response can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Positive clinical response may also be expressed in terms of various measures of clinical outcome. Positive clinical outcome can also be considered in the context of an individual's outcome relative to an outcome of a population of patients having a comparable clinical diagnosis, and can be assessed using various endpoints such as an increase in the duration of recurrence-free interval (RFI), an increase in the time of survival as compared to overall survival (OS) in a population, an increase in the time of disease-free survival (DFS), an increase in the duration of distant recurrence-free interval (DRFI), and the like. Additional endpoints include a likelihood of any event (AE)-free survival, a likelihood of metastatic relapse (MR)-free survival (MRFS), a likelihood of disease-free survival (DFS), a likelihood of relapse-free survival (RFS), a likelihood of first progression (FP), and a likelihood of distant metastasis-free survival (DMFS). An increase in the likelihood of positive clinical response corresponds to a decrease in the likelihood of cancer recurrence or relapse.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, “synergistic” means more than additive. Synergistic effects may be measured by various assays known in the art.

As used herein, the term “toxin” means an antigenic poison or venom of plant or animal origin. An example is diphtheria toxin or portions thereof.

The term “treatment,” “treat,” or “treating” refers to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%, 80%, 90%, or 100% reduction in the severity of a cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

### Neutrophil Related Biomarker

The present disclosure in one aspect provides a method of treating cancer patients with a combinational immunotherapy based on a neutrophil related biomarker that can predict the responsiveness of the combinational immunotherapy. In one embodiment, the method comprises: measuring a base level of the neutrophil related biomarker in a sample from the subject; determining that the base level of said biomarker is equal or more than a threshold value; and administering to the subject a combinational immunotherapy.

Neutrophils, also known as neutrocytes or polymorphonuclear myeloid-derived suppressor cells (PMN-MDSCs), are a type of phagocyte normally found in the bloodstream. In most mammals, neutrophils are the most abundant type of granulocytes and the most abundant type of white blood cell. Neutrophils form an essential part of the innate immune system and play various functions in different contexts. During an acute inflammation, particularly as a result of bacterial infection and some cancers, neutrophils are one of the first-responders of inflammatory cells to migrate to the site of inflammation.

Methods of detecting and measuring the number of neutrophils are known in the art. For example, hematoxylin and eosin (H&E) staining has long been used to differentiate neutrophils from basophilic and eosinophilic white blood cells. Neutrophils can also be identified by the expression of certain markers, e.g., CD11c, CD13, CD15, CD16, CD33 and CD68.

Myeloid derived suppressor cells (MDSCs) are a heterogeneous group of immature myeloid cells which suppress the immune system. Collectively a MDSC population is comprised of monocyte-like MDSCs and polymorphonuclear MDSC (PMN-MDSCs or neutrophils). The number of MDSCs is increased with the presence of tumors. It has been shown that PMN-MDSCs represent the majority of MDSCs in cancers and protect the cancers from the immune system.

As used herein, the term “neutrophil related biomarkers” refer to biomarkers that are indicative of the presence, abundance or activation of neutrophils in any sample or tissue of the subject. In certain embodiments, the neutrophil related biomarker is selected from a group consisting of hepatocyte growth factor, absolute neutrophil count, c-Met+ neutrophils and neutrophil to lymphocyte ratio (NLR).

In certain embodiments, the neutrophil related biomarker is NLR and the threshold value is about 3, 3.5, 4, 4.5 or 5.

In another embodiment, the method comprises: measuring a first level of the biomarker in the subject; administering to the subject an immunotherapy for a time period; measuring a second level of the biomarker in the subject; determining that a difference between the second level of the biomarker and the first level of biomarker is equal or more than a critical value; and administering to the subject a combinational immunotherapy.

In certain embodiments, wherein the neutrophil related biomarker is NLR and the critical value is about 2, 2.5, 3, 3.5 or 4.

In certain embodiments, the subject being treated is a mammal. In certain embodiments, the mammal is selected from the group consisting of humans, primates, farm animals and domestic animals. In certain embodiments, the mammal is a human.

In certain embodiment, the cancer being treated is selected from the groups consisting of a lung cancer, a melanoma, a renal caner, a liver cancer, a myeloma, a prostate cancer, a breast cancer, a colorectal cancer, a pancreatic cancer, a thyroid cancer, a hematological cancer, a leukemia and a non-Hodgkin's lymphoma.

### Combinatorial Usage of c-Met Inhibitor and Modulators of Immune Checkpoint

In another aspect, the present disclosure provides a method of treating cancer using a combination immunotherapy. In certain embodiments, when it is determined that the subject is likely responsive to a combinational immunotherapy, e.g., by monitoring the neutrophil related biomarker as discussed above, the combinational immunotherapy is administered to the subject. In certain embodiment, the combinational immunotherapy is a combination use of a c-Met inhibitor and a modulator of an immune checkpoint. In some embodiments, the modulator of an immune checkpoint is an anti-PD-1 antibody or an anti-PD-L1 antibody.

c-MET is a proto-oncogene that encodes a protein known as hepatocyte growth factor receptor (HGFR). c-Met protein is composed of the a chain and β chain generated by cleaving a precursor of c-Met (pro c-Met) and forms a dimer by a disulfide linkage. c-Met is a receptor penetrating a cell membrane and the entire a chain and a part of the β chain are present extracellularly (see, e.g., Mark, et al., The Journal of Biological Chemistry, 1992, Vol. 267, No. 36, pp. 26166-26171; Journal of Clinical and Experimental Medicine (IGAKU NO AYUMI), 2008, Vol. 224, No. 1, pp. 51-55). See also GenBank Accession No: NP_000236.2 for human c-Met and its α chain and β chain. It has been shown that abnormal MET activation in cancer correlates with poor prognosis, where aberrantly active c-Met triggers tumor growth, formation of new blood vessels that supply the tumor with nutrients, and cancer spread or other organs.

A “c-Met inhibitor,” as used herein, refers an agent that can suppress the expression or activity of c-Met protein. In certain embodiments, c-Met inhibitor is selected from the group consisting of crizotinib, cabozantinib, APL-101, PLB1001, bozitinib, SU11274, PHA665752, K252a, PF-2341066, AM7, JNJ-38877605, PF-04217903, MK2461, GSK1363089 (XL880, foretinib), AMG458, tivantinib (ARQ197), INCB28060 (INC280, capmatinib), E7050, BMS-777607, savolitinib (volitinib), HQP-8361, merestinib, ARGX-111, onartuzumab, rilotumumab, emibetuzumab, and XL184.

In some embodiments, the c-Met inhibitor comprises a compound of the following formula

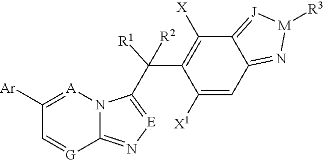

wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl;

In some embodiments, the c-Met inhibitor is selected from the group consisting of:

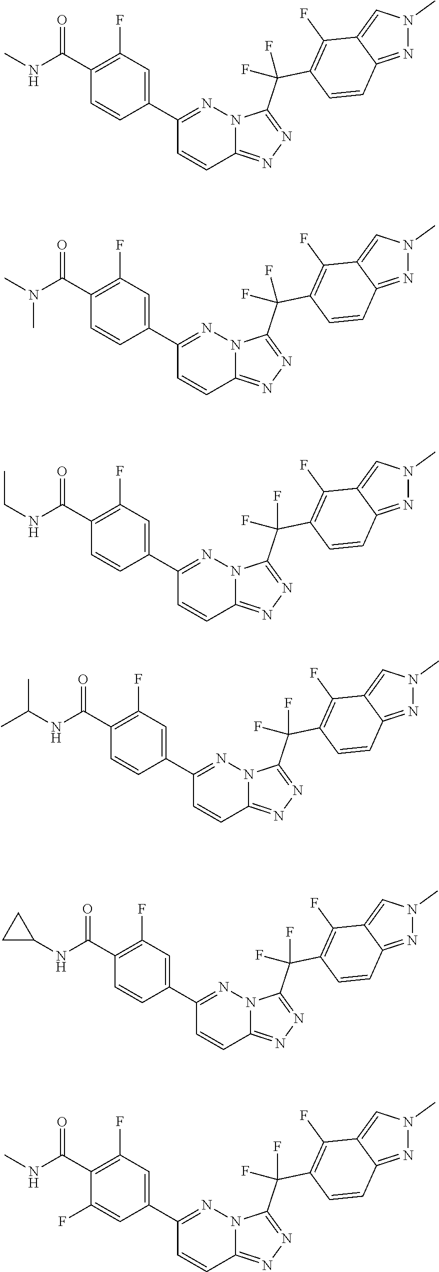

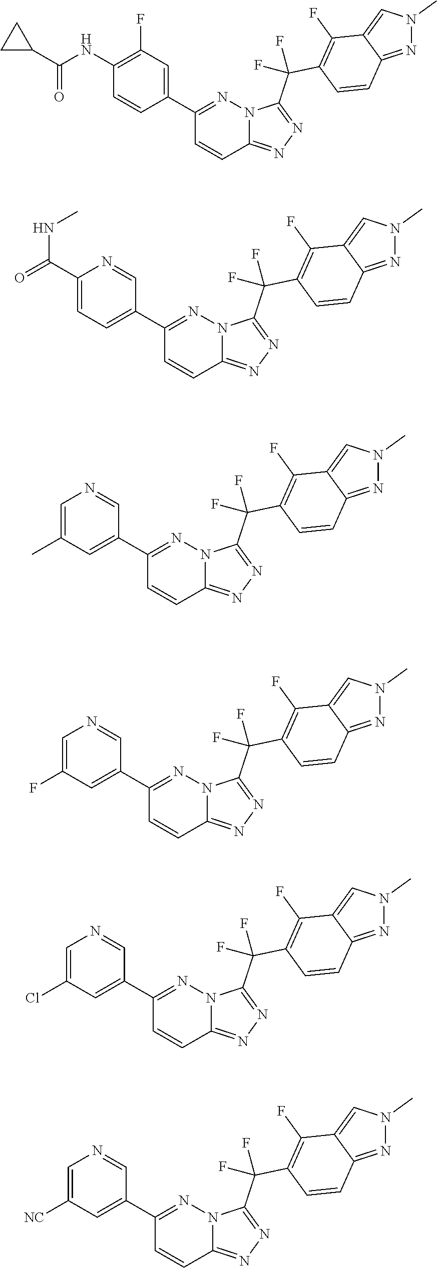

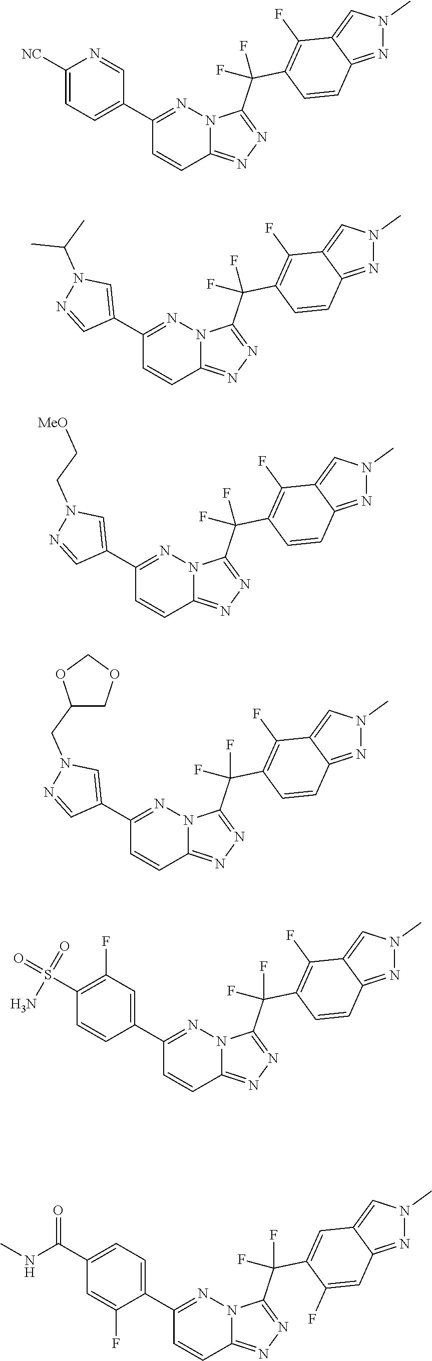

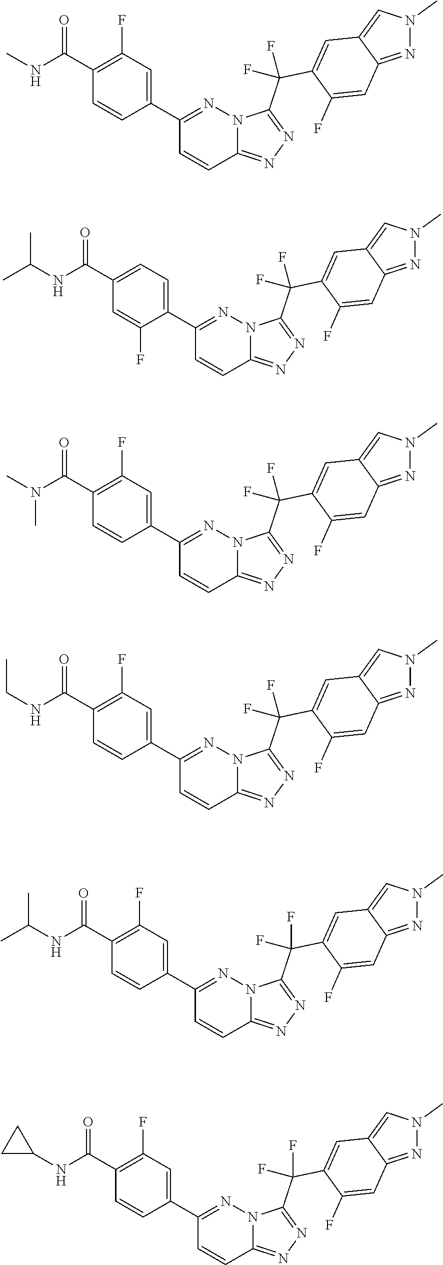

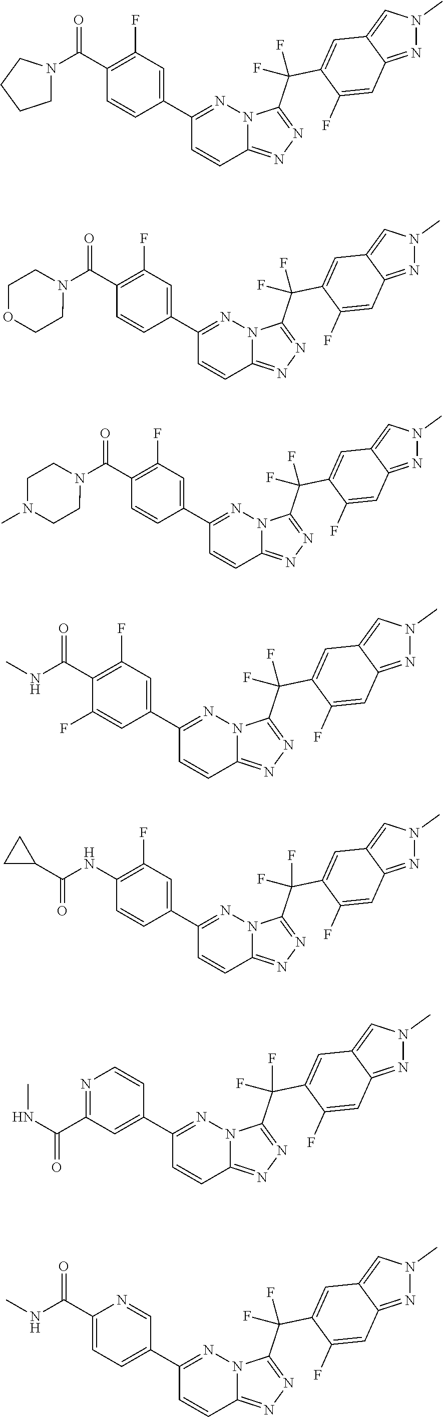

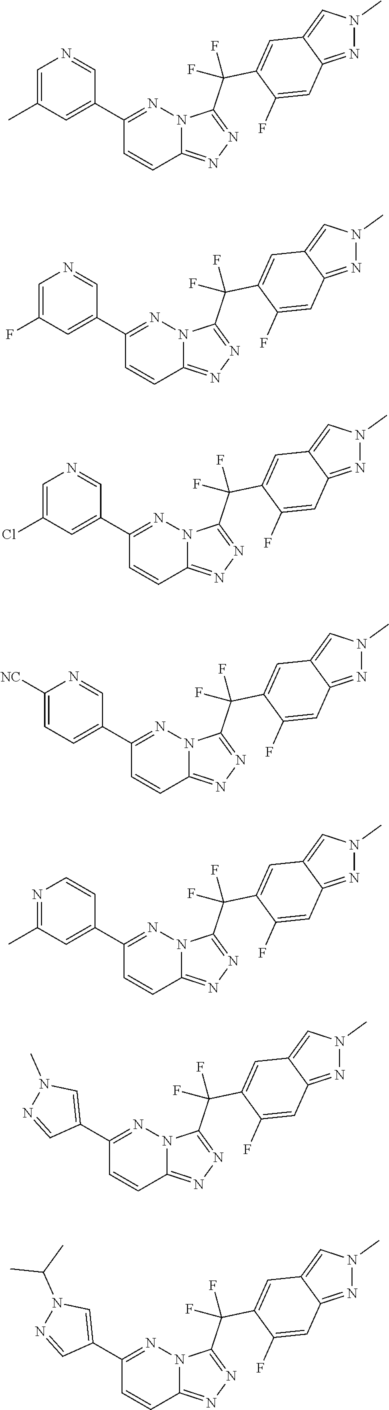

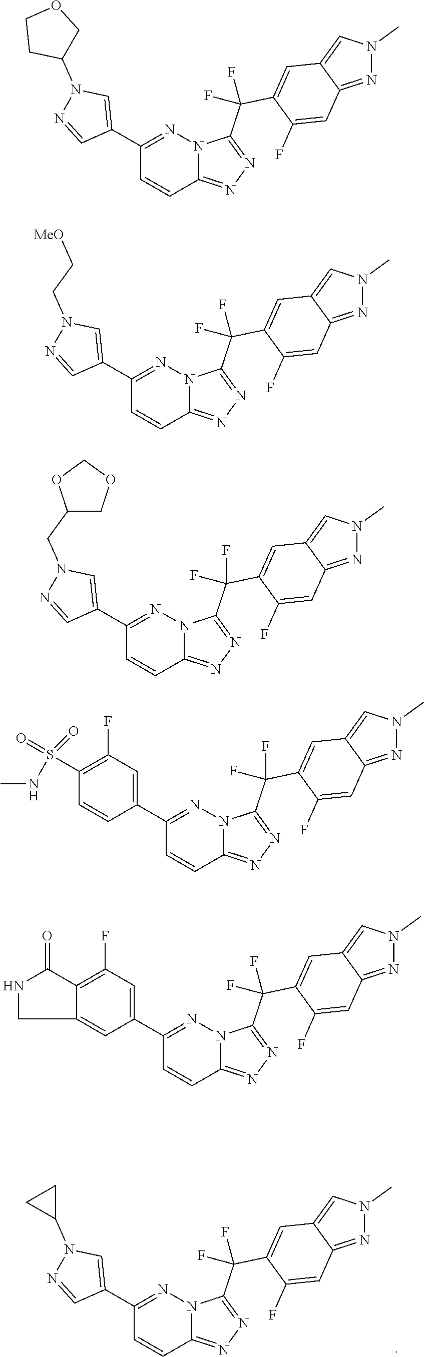

In certain embodiments, c-Met inhibitor is APL-101 (previously named CBT-101, see US20150218171, which is incorporated in its entirety by reference), which has the following formula:

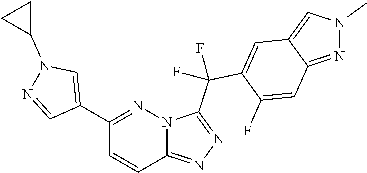 [SMILES: Cn1cc2cc(C(F)(F)c3nnc4ccc(-c5cnn(C6CC6)c5)nn34)c(F)cc2n1]

In certain embodiments, c-Met inhibitor can be formulated with a pharmaceutically acceptable carrier. The carrier, when present, can be blended with c-Met inhibitor in any suitable amounts, such as an amount of from 5% to 95% by weight of carrier, based on the total volume or weight of c-Met inhibitor and the carrier. In some embodiments, the amount of carrier can be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%. The amount of carrier in a specific embodiment may be determined based on considerations of the specific dose form, relative amounts of c-Met inhibitor, the total weight of the composition including the carrier, the physical and chemical properties of the carrier, and other factors, as known to those of ordinary skill in the formulation art.

As used herein, the term “immune checkpoint” or “cancer immune checkpoint” refers to a molecule in the immune system that either turns up a signal (i.e., co-stimulatory molecules) or turns down a signal (i.e., inhibitory molecule) of an immune response. In certain embodiments, the immune checkpoint is selected from the group consisting of PD-1, PD-L1, PD-L2, LAG-3, TIM-1, CTLA-4, VISTA, B7-H2, B7-H3, B7-H4, B7-H6, 284, ICOS, HVEM, CD160, gp49B, PIR-B, KIR family receptors, TIM-1, TIM-4, BTLA, SIRPalpha (CD47), CD48, 284 (CD244), B7.1, B7.2, ILT-2, ILT-4, TIGIT and A2aR.

In certain embodiments, the modulator of immune checkpoint is a monoclonal antibody against the immune checkpoint. In certain embodiments, the immune checkpoint is PD-1 or PD-L1. In certain embodiments, the anti-PD-1 antibody is selected from those disclosed in PCT application publication No. WO2016/014688, which is incorporated in its entirety by reference. In certain embodiments, the anti-PD-1 antibody is APL-501 (previously named as CBT-501, see WO2016/014688), GB226 or genolimzumab. In certain embodiments, the anti-PD-L1 antibody is selected from those disclosed in PCT application publication No. WO2016/022630, which is incorporated in its entirety by reference. In certain embodiments, the anti-PD-L1 antibody is APL-502 (previously named as CBT-502, see WO2016/022630) or TQB2450.

According to the present disclosure, the c-Met inhibitor and the modulator of immune checkpoint (or another anti-cancer therapeutic agent) may be co-administered to the subject, either simultaneously or at different times, as deemed most appropriate by a physician. If the c-Met inhibitor and the immune checkpoint modulator are administered at different times, for example, by serial administration, the immune checkpoint modulator may be administered to the subject before the c-Met inhibitor. Alternatively, the c-Met inhibitor may be administered to the subject before immune checkpoint modulator.

The c-Met inhibitor or the modulator of immune checkpoint or other anti-cancer therapeutic agents may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the c-Met inhibitor or the modulator of immune checkpoint or other anti-cancer therapeutic agents may be administered in conjunction with other treatments. The c-Met inhibitor or the modulator of immune checkpoint or other anti-cancer therapeutic agents may be encapsulated or otherwise protected against gastric or other secretions, if desired.

A suitable, non-limiting example of a dosage of the c-Met inhibitor or the modulator of immune checkpoint or other anti-cancer therapeutic agents disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. In some embodiments, the dosage of the c-Met inhibitor in human is about 400 mg/day given every 12 hours. In some embodiments, the dosage of the c-Met inhibitor in human ranges 300-500 mg/day, 100-600 mg/day or 25-1000 mg/day. The effective dose of c-Met inhibitor or the modulator of immune checkpoint or other anti-cancer therapeutic agents disclosed herein may be administered as two, three, four, five, six or more sub-doses, administered separately at appropriate intervals throughout the day.

### Other Combinational Therapies

In one embodiment, the method further comprises administering at least one additional therapeutic agent selected from the group consisting of a cytotoxic agent, a toxin, a radionuclide, an immunomodulator, a photoactive therapeutic agent, a radiosensitizing agent, a hormone, an anti-angiogenesis agent, and combinations thereof. In certain embodiments, the administration of the c-Met inhibitor, the modulator of immune checkpoint and the additional therapeutic agent provides a synergistic effect.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLE 1

This example illustrates the synergic effect of combination treatment using a c-Met inhibitor (APL-101) and an anti-PD-1 antibody in MC-38 syngeneic colon cancer model.

### Experimental Design

The inventors undertook a combination study of APL-101 and an anti-PD-1 antibody to evaluate the safety and efficacy of the combination. In the MC-38 colon cancer model in syngeneic mice, four groups, five animals per group received either vehicle (water at 20 mg/kg orally, once a day), APL-101 (10 mg/kg orally, once a day), anti-PD-1 (10 mg/kg intraperitoneal injection, twice a week), or APL-101 plus anti-PD-1. In the vehicle group as well as the APL-101 group, animals were dosed daily on Days 1-15 whereas in the single agent anti-PD-1 group, doses were administered on Days 1, 4, 8, 11, and 15. In the combination arm of APL-101 and anti-PD-1, APL-101 was administered on Days 5-15 (4-day delay) while the anti-PD-1 was dosed on Days 1, 4, 8, 11, and 15.

### Materials and Methods

Animals: female C57BL/6 mice, age 6-8 weeks and of body weight 18-20 g, were provided by Shanghai Lingchang Bio-Technology Co. Ltd.

APL-101 were provided by CBT pharmaceuticals (now Apollomics, Inc.). Anti-PD 1 antibodies were supplied by BioXcell.

Cell culture: The MC38 tumor cells were thawed and maintained in vitro as a monolayer culture in DMEM medium supplemented with 10% heat inactivated fetal bovine serum at 37° C. in an atmosphere of 5% CO2 in air. The tumor cells were routinely subcultured twice weekly by trypsin-EDTA treatment. The cells growing in an exponential growth phase were harvested and counted for tumor inoculation.

Tumor inoculation: Each mouse was inoculated subcutaneously at the right lower flank with MC38 tumor cells (1×10⁶) in 0.1 ml of PBS. The treatments started when the mean tumor size reached approximately 80-120 mm³. The date of tumor cell inoculation is denoted as day 0.

Group assignment: Before grouping and treatment, all animals were weighed and the tumor volumes were measured using a caliper. Since the tumor volume can affect the effectiveness of any given treatment, tumor volume was used as numeric parameter to randomize selected animals into specified groups. The grouping was performed by using StudyDirector™ software (Studylog Systems, Inc. CA, USA).

Observation and data collection: After tumor cells inoculation, the animals were checked daily for morbidity and mortality. During routine monitoring, the animals were checked for any effects of tumor growth and treatments on normal behavior such as mobility, visual estimation of food and water consumption, body weight gain/loss (body weights were measured twice per week after randomization), eye/hair matting and any other abnormal effect. Death and observed clinical signs were recorded in the comment of datasheet for each animal in detail. Tumor volumes were measured twice weekly after randomization in two dimensions using a caliper, and the volume was expressed in mm³ using the formula: V=0.5 a×b² where a and b are the length and width of the tumor, respectively. (Tumor weight was measured at the end of study). The entire procedures of dosing as well as tumor and body weight measurement were conducted in a Laminar Flow Cabinet.

Statistics: the mean and standard error of the mean (SEM) were provided for the tumor volumes of each group at every time point. Statistical analysis of difference in tumor volume between the two comparing groups was conducted on the data obtained at the best therapeutic time point (usually after the final dose) using One-way ANOVA Test. All data were analyzed in SPSS (Statistical Product and Service Solutions) version 18.0 (IBM, Armonk, N.Y., U.S.). P-values were rounded to three decimal places, with the exception when raw P-values were less than 0.001, then they were stated as P<0.001. All tests were two-sided. P<0.05 was considered to be statistically significant.

### Results

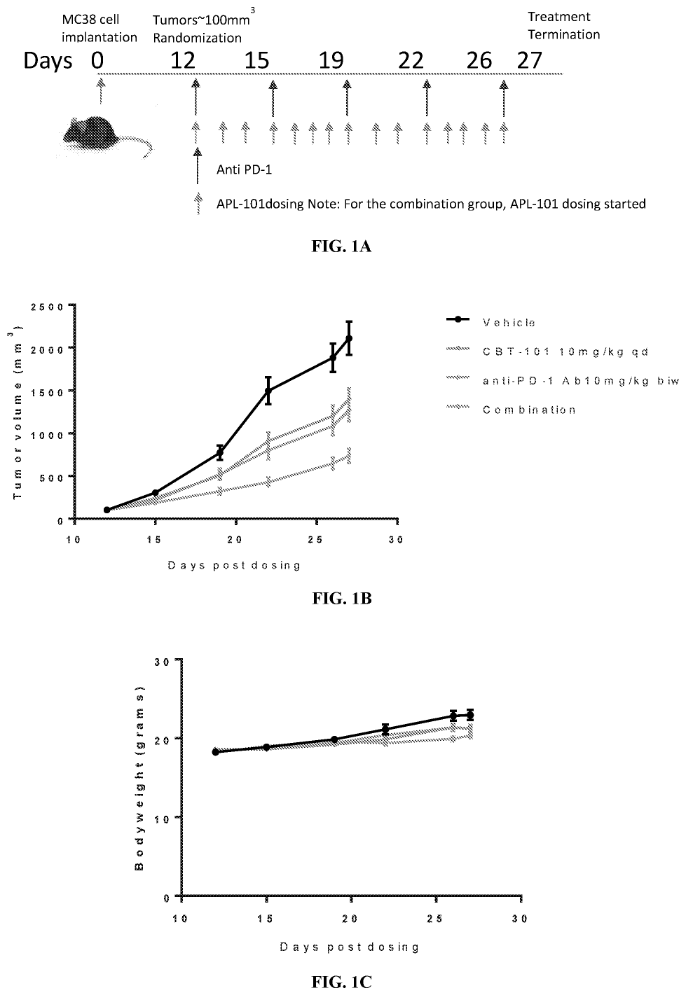

As shown in FIGS. 1A-1C and Table 1, mean percent tumor growth inhibition of the combination anti-PD-1 10 mg/kg IP BIW×2 weeks plus APL-101 10 mg/kg, QD×2 weeks demonstrated a 65.1% tumor growth inhibition, versus 39.9% and 33.6% for anti-PD-1 IP 10 mg/kg BIW×3 weeks and APL-101 PO 10 mg/kg, QD×3 weeks, respectively. The combination regimen was well tolerated by the animals. Tumor tissue collected for c-Met positivity and PD-L1 neutrophils is evaluated along with neutrophil to lymphocyte ratio.

TABLE 1
Mean percent tumor growth in MC 38 syngeneic model.

| Vehicle | |
|---|---|
| APL-101 10 mg/kg qd | 35.61 |
| Anti-PD-1 Ab 10 mg/kg biw | 42.06 |
| Combination | 23.97 |

### EXAMPLE 2

This example illustrates the synergic effect of combination treatment using a c-Met inhibitor (APL-101) and an anti-PD-1 antibody in H22 syngeneic liver cancer model.

### Experimental Design

The inventors undertook a combination study of APL-101 and an anti-PD-1 antibody to evaluate the safety and efficacy of the combination. In the H22 liver cancer model in syngeneic mice, four groups, ten animals per group received either vehicle (PVP K30 at 20 mg/kg orally, once a day for three weeks), APL-101 (10 mg/kg orally, once a day for three weeks), anti-PD-1 (10 mg/kg intraperitoneal injection, twice a week for three weeks), or APL-101 plus anti-PD-1.

### Materials and Methods

Animals: female C57BL/6 mice, age 6-8 weeks and of body weight 18-20 g, were provided by Shanghai Lingchang Bio-Technology Co. Ltd.

APL-101 were provided by CBT pharmaceuticals (now Apollomics, Inc.). Anti-PD 1 antibodies were supplied by BioXcell. PVP K30 were supplied by Fluka Analytical.

Cell culture: The H22 tumor cell line were maintained in vitro in RPMI-1640 medium supplemented with 10% fetal bovine serum at 37° C. in an atmosphere of 5% CO₂ in air. The tumor cells were routinely subcultured twice weekly by trypsin-EDTA treatment. The cells growing in an exponential growth phase were harvested and counted for tumor inoculation.

Tumor inoculation: Each mouse was inoculated subcutaneously at the right front flank with H22 tumor cells (2×10⁶) in 0.1 ml of PBS for tumor development. The treatments were started when the mean tumor size reaches approximately 80-120 mm³. The date of tumor cell inoculation was denoted as day 0.

Randomization: The randomization started when the mean tumor size reached approximately 80-120 mm³. 40 mice were enrolled in the study. All animals were randomly allocated to 4 study groups. Randomization was performed based on randomized block design.

Observation and data collection: After tumor cells inoculation, the animals were checked daily for morbidity and mortality. During routine monitoring, the animals were checked for any effects of tumor growth and treatments on behavior such as mobility, food and water consumption, body weight gain/loss (body weights were measured twice weekly after randomization), eye/hair matting and any other abnormalities. Mortality and observed clinical signs were recorded for individual animals in detail. Tumor volumes were measured twice weekly in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). (Tumor weight were measured at the end of study). Dosing as well as tumor and body weight measurements were conducted in a Laminar Flow Cabinet.

Statistics analysis: For comparison among three or more groups, a one-way ANOVA was performed followed by multiple comparison procedures. For survival analysis, Kaplan-Meier survival curves was generated and Log Rank test was performed. All data was analyzed using SPSS 18.0. P<0.05 was considered statistically significant.

### Results

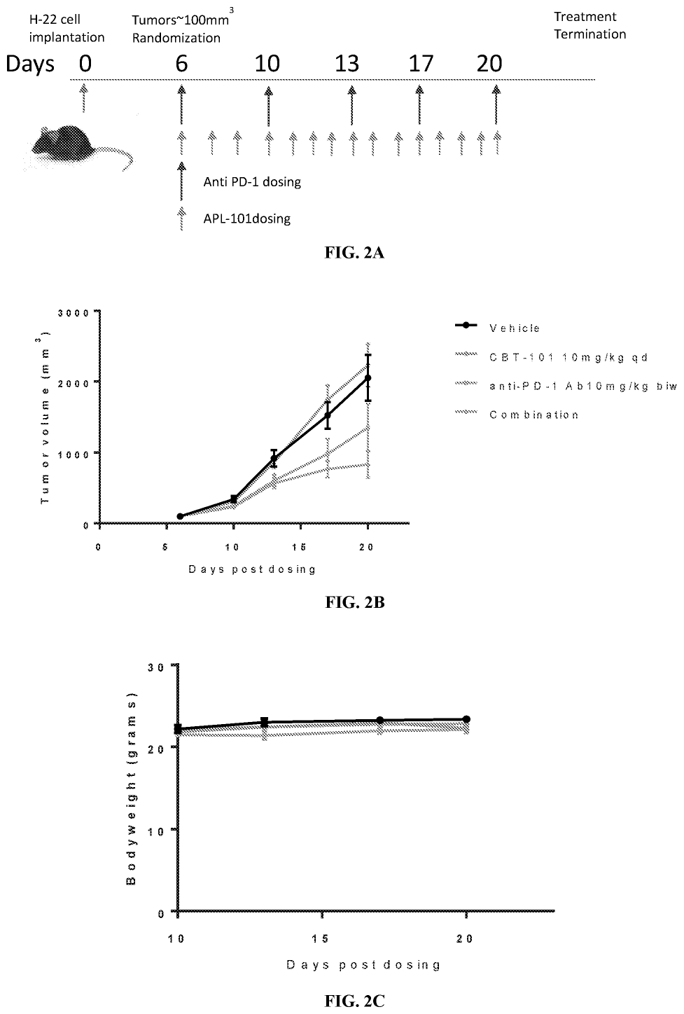

As shown in FIGS. 2A-2C and Table 2, mean percent tumor growth of the combination anti-PD-1 10 mg/kg IP BIW×3 weeks plus APL-101 10 mg/kg, QD×3 weeks demonstrated a 40.38% tumor growth, versus 108.73% for APL-101 10 mg/kg, QD×3 weeks and 65.85% for anti-PD-1 IP 10 mg/kg BIW×3 weeks, respectively. The combination regimen was well tolerated by the animals.

TABLE 2
Mean percent tumor growth in H22 syngeneic liver cancer model.

| Vehicle | |
|---|---|
| APL-101 10 mg/kg qd | 108.73 |
| Anti-PD-1 Ab 10 mg/kg biw | 65.85 |
| Combination | 40.38 |

### EXAMPLE 3

This example illustrates the synergic effect of combination treatment using a c-Met inhibitor (APL-101) and an anti-PD-1 antibody in a syngeneic Renca kidney cancer model.

### Experimental Design

The inventors undertook a combination study of APL-101 and an anti-PD-1 antibody to evaluate the safety and efficacy of the combination. In the Renca kidney cancer model in syngeneic mice, four groups, ten animals per group received either vehicle (PVP K30 at 20 mg/kg orally, once a day for three weeks), APL-101 (20 mg/kg orally, once a day for three weeks), anti-PD-1 (10 mg/kg intraperitoneal injection, twice a week for three weeks), or APL-101 (20 mg/kg orally, once a day for three weeks) plus anti-PD-1 (10 mg/kg intraperitoneal injection, twice a week for three weeks).

### Materials and Methods

Animals: female C57BL/6 mice, age 6-8 weeks and of body weight 18-20 g, were provided by Shanghai Lingchang Bio-Technology Co. Ltd.

APL-101 were provided by CBT pharmaceuticals (Apollomics, Inc.). Anti-PD 1 antibodies were supplied by BioXcell. PVP K30 were supplied by Fluka Analytical.

Cell culture: The Renca tumor cell line was maintained in vitro in DMEM medium supplemented with 10% fetal bovine serum at 37° C. in an atmosphere of 5% CO₂ in air. The tumor cells were routinely subcultured twice weekly. The cells growing in an exponential growth phase were harvested and counted for tumor inoculation.

Tumor inoculation: Each mouse was inoculated subcutaneously at the right front flank with RENCA tumor cells (1×10⁶) in 0.1 ml of PBS for tumor development. The treatments were started when the mean tumor size reaches approximately 80-120 mm³. The date of tumor cell inoculation was denoted as day 0.

Randomization: The randomization started when the mean tumor size reached approximately 80-120 mm³. 40 mice were enrolled in the study. All animals were randomly allocated to 4 study groups. Randomization was performed based on randomized block design.

Observation and data collection: After tumor cells inoculation, the animals were checked daily for morbidity and mortality. During routine monitoring, the animals were checked for any effects of tumor growth and treatments on behavior such as mobility, food and water consumption, body weight gain/loss (body weights were measured twice weekly after randomization), eye/hair matting and any other abnormalities. Mortality and observed clinical signs were recorded for individual animals in detail. Tumor volumes were measured twice weekly in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). (Tumor weight were measured at the end of study). Dosing as well as tumor and body weight measurements were conducted in a Laminar Flow Cabinet.

Statistics analysis: For comparison among three or more groups, a one-way ANOVA was performed followed by multiple comparison procedures. For survival analysis, Kaplan-Meier survival curves was generated and Log Rank test was performed. All data was analyzed using SPSS 18.0. P<0.05 was considered statistically significant.

### Results

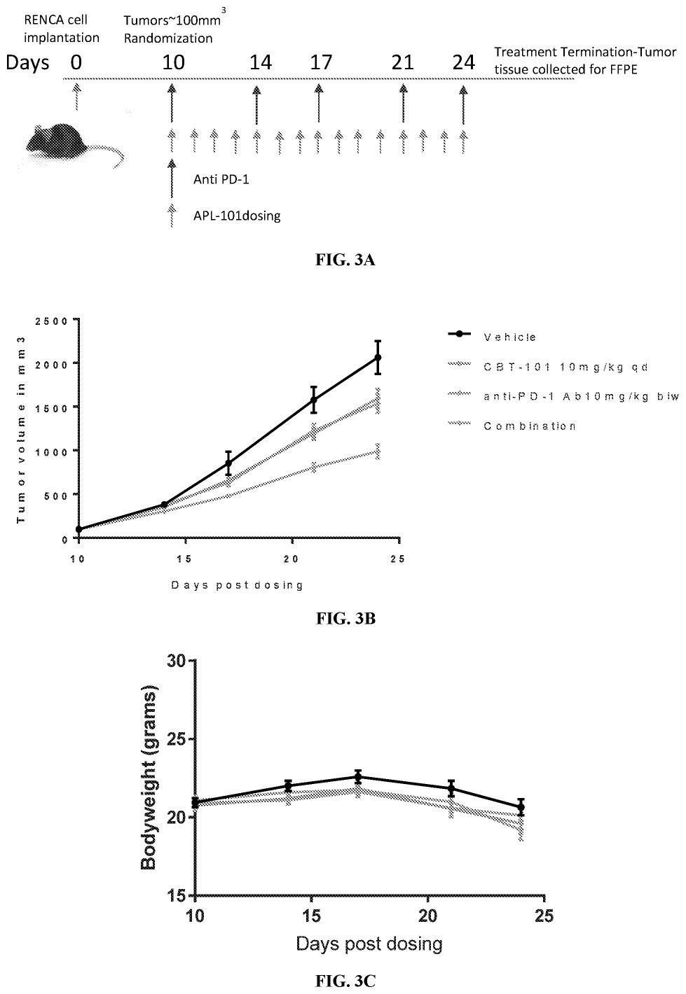

As shown in FIGS. 3A-3C and Table 3, mean percent tumor growth of the combination anti-PD-1 10 mg/kg IP BIW×3 weeks plus APL-101 10 mg/kg, QD×3 weeks demonstrated a 47% tumor growth, versus 77% for APL-101 10 mg/kg, QD×3 weeks and 71% for anti-PD-1 IP 10 mg/kg BIW×3 weeks, respectively. The combination regimen was well tolerated by the animals.

TABLE 2
Mean percent tumor growth in syngeneic
Renca kidney cancer model.

| Vehicle | |
|---|---|
| APL-101 10 mg/kg qd | 77 |
| Anti-PD-1 Ab 10 mg/kg biw | 71 |
| Combination | 47 |

### EXAMPLE 4

This example illustrates that a combination of c-Met inhibitor (APL-101) and an anti-PD-1 antibody deceased the neutrophil percentage in tumor microenvironment.

### Experimental Design

Tumor tissues was collected from the MC38 colon adenocarcinoma syngeneic model (described in Example 1) at the end of the study and fixed in formalin. Double IHC analysis of c-Met and neutrophils was used to quantify the expression of Met+ neutrophils.

Sample preparation: fresh specimens were collected and placed in 10% NBF (neutral-buffered formalin; fixative volume/tissue, 10˜20 folds), fixed at room temperature for 24 hours. Fixed tissue was trimmed at the thickness of 3-5 mm. The trimmed tissues were moved into an embedding box. The box was snapped into deionized water for 30 minutes, with water changed twice every 30 minutes. If the dehydration procedure could not be carried out on time, the tissues were transferred into the 70% ethanol, and placed in the 4° C. refrigerator. The tissues can be kept in 70% ethanol for about 3-5 days in the refrigerator. After dehydration, FFPE preparation and FFPE slide preparation of the fixed tissues were transferred to the LEICA ASP300S Vacuum Tissue Processor for dehydration.

FFPE slides preparation: The dehydrated tissues were be embedded in paraffin on Paraffin Embedding Station. The FFPE blocks were sectioned with a manual rotary microtome, 4 μm thickness/section.

The FFPE slides were used for IHC with the following antibodies: anti-neutrophil (LY6G/C) (abcam Cat # ab2557); anti-c-Met (abcam Cat # ab51067); goat anti-Rb IgG (Leica Cat # DS9800); anti-Rat IgG (vector Cat # MP-7444-15).

Image scan: All stained sections were scanned with NanoZoomer-HT 2.0 Image system for 40× magnification (Hamamatsu photonics) with 3 fluorescence channels: Red, Green, Blue. High resolution picture for whole section were generated and further quantification analysis.

Score for IHC staining: The first step was to take an overall look the staining pattern and to exclude the necrosis and big stroma areas. Five representative fields were chosen from each sample to do quantification analysis. Five fields in each staining were selected and imaged at 20× magnification. All the images were analyzed with Image J software. c-Met and Ly6G/C co-localized cells and total cells were counted. Double IF scores were presented as the ratio of the average of the c-Met and Ly6G/C co-localized cell counts against the total cell numbers in the five fields.

### Results

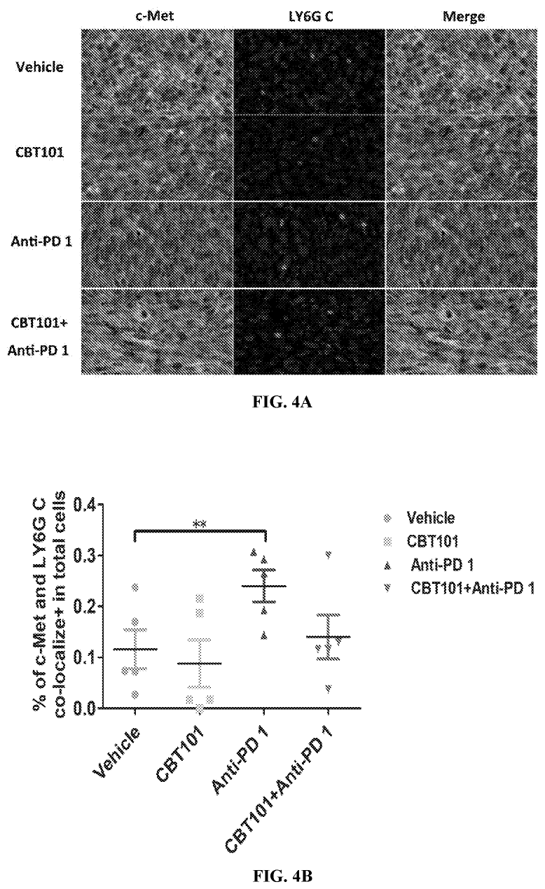

As shown in FIGS. 4A-4B, anti-PD1 antibody increased c-Met positive neutrophils, and anti-PD1 plus c-Met inhibitor decreased the neutrophil percentage in tumor microenvironment. As shown in FIG. 4C, a treatment of anti-PD-1 antibody increased c-Met positive neutrophils in peripheral circulation, and a combination of a c-Met inhibitor and an anti-PD-1 antibody decreased the neutrophil percentage in peripheral circulation.

### EXAMPLE 5

This example illustrates the evaluation of in vivo efficacy of c-Met inhibitor and anti-PD-1 antibodies in NSCLC, RCC, HCC and Gastric cancer patients.

A combination trial is designed to find the subset of patients that are unlikely to benefit from PD-1 single agent therapy (e.g., HCC and RCC) due to infiltration of c-Met⁺ neutrophils in tumor, and co-administration of a c-Met inhibitor with PD-1 is expected to restore the full PD-1 effect in this population. Combination treatment with a c-Met inhibitor with a PD-1 inhibitor could form a bridge between T cells and tumor cells, allowing the T cells to target the tumor cells directly. With these distinct mechanisms of action, APL-101 (c-Met inhibitor) and APL-501 (anti-PD-1 antibody) combination treatment acts synergistically in enhancing the host anti-tumor response.

In Cycle 1, Day 1, starting in the evening, APL-101 is administered concomitantly with the PD-1 inhibitors administered continuously (Day 1-Day 28) throughout the 28-day cycle. This allows to test if a blood biomarker can predict the population studied—neutrophil or HGF—either at baseline or change upon PD-1 single agent treatment. Neutrophil to lymphocyte ratio, platelet to lymphocyte ratio, HGF and other markers have been postulated as predictive biomarkers for PD-1 non-response in HCC, mRCC, and other tumors (e.g., NSCLC).

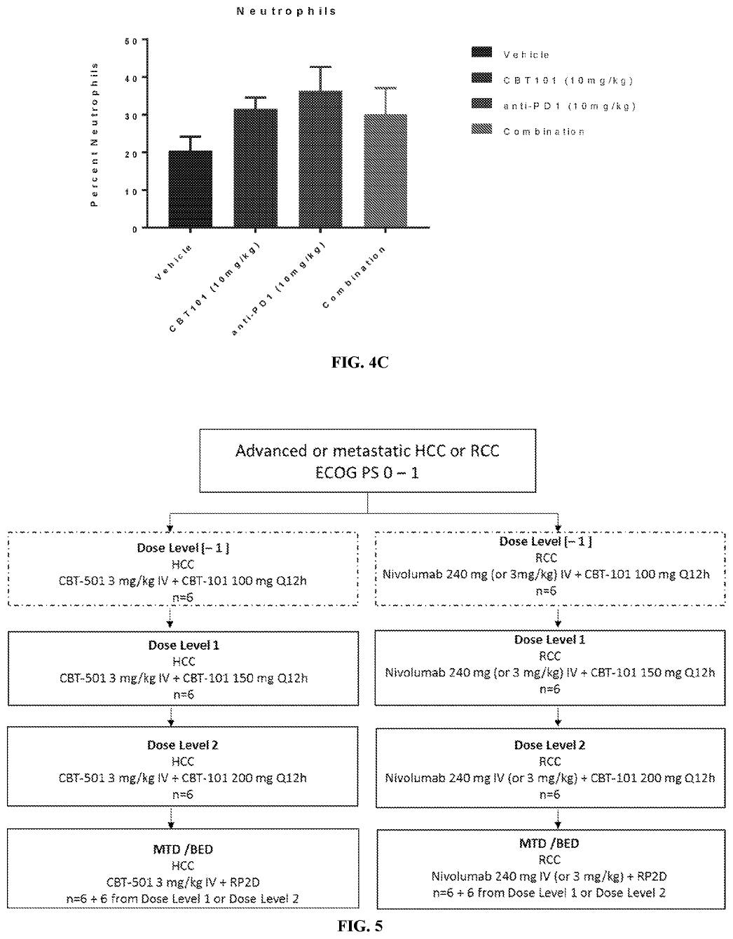

As illustrated in FIG. 5, in the Phase 1 portion, eligible HCC and RCC subjects receive APL-501 intravenously (IV) or nivolumab IV on Day 1 and Day 15 on a 28-day cycle and APL-101 orally every 12 hours for 28 consecutive days of each 28-day cycle. The dose of APL-501 at 3 mg/kg administered intravenously on Day 1 and Day 15 of a 28-day cycle is based on an ongoing Phase 1 clinical trial in Australia with relapsed and refractory select solid tumor subjects. Nivolumab 240 mg or 3 mg/kg every 2 weeks administration (Day 1 and Day 15) is based on the approved label for the US or Australia/New Zealand, respectively. The PD-1 inhibitor doses is fixed. The APL-101 dose is escalated or de-escalated pending toxicities. APL-101 starting dose is based on (150 mg every 12 hours; 300 mg total daily dose) is based on clinical data from ongoing clinical trials in China with APL-101 (NCT02896231 and NCT02978261). In each instance, the Safety Review Committee has deemed the 3 mg/kg and 300 mg dose as safe for APL-501 and APL-101, respectively. The trial is designed to find a safe dose combination (R2PD) of APL-501+APL-101 primarily and nivolumab+APL-101 secondarily.

If two or more DLTs occur among 6 subjects in a cohort, then enrollment into that cohort is stopped and the previous dose level is considered the tentative MTD. All 6 additional subjects in the tentative MTD group must complete one cycle of combination PD-1 plus APL-101 administration. Subjects who drop out before they complete the first cycle of treatment for reasons other than toxicity are replaced. Dose escalation to Dose Level 2 is only allowed after review and approval of the SRC of all Cycle 1 safety data. The SRC evaluates the overall tolerability of combination therapy (e.g., sustained Grade 2 adverse events, dose reductions, and dose interruptions and any occurrences of delayed toxicities) prior to recommending the RP2D for further evaluation. Once the RP2D has been determined, intra-patient dose escalation is permitted for subjects enrolled at lower doses that continue to receive clinical benefit from PD-1 plus APL-101 and may be escalated to the RP2D. PK sampling and evaluation occurs in Phase 1 for all cohorts levels evaluated.

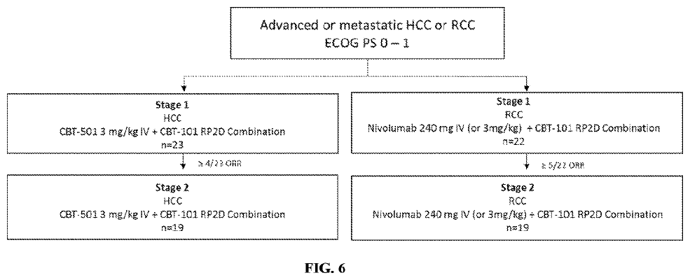

Phase 2 confirms safety, tolerability and efficacy of the RP2D as determined in Phase 1 in subjects with locally advanced and metastatic HCC and RCC. As illustrated in FIG. 6, based on Simon Minimax design, the recommended APL-101 Phase 2 dosed is further evaluated in twenty-three and twenty-two HCC and RCC subjects respectively. If the ORR demonstrates ≥4 responses of the 23 subjects enrolled in Stage 1 of the HCC arm, an additional 19 subjects are enrolled in Stage 2. Similarly, if the ORR demonstrates ≥5 responses of the 23 subjects enrolled in Stage 1 of the RCC arm, an additional 19 subjects are enrolled in Stage 2. No PK sampling and evaluation occurs in Phase 2.

For each potential subject, there is a 28-day screening and eligibility assessment period before enrollment; the first dose of study treatment is administered on Day 1 of Cycle 1 (C1D1) (Safety and Intent-to-Treat population). Subjects continue to receive their assigned treatment throughout the study until the occurrence of confirmed disease progression [progressive disease (PD)] by irRECIST, and secondarily by mRECIST for HCC subjects, death, unacceptable treatment-related toxicity, or until the study is closed by the Sponsor. During the treatment period, study visits occur on Day 1, Day 2, Day 8, Day 15, and Day 16 during Cycle 1 and Day 1 and Day 15 of every subsequent cycle. Subjects who experience a response [Complete Response (CR), Partial Response (PR)]≥2 cycles, PD-1 plus APL-101 combination is continued for at least 2 additional cycles beyond response. Subjects receive a minimal of 2 cycles of PD-1 and APL-101 for adequate evaluation of response (Evaluable population). Discontinuation of PD-1 and APL-101 occurs upon determination of progressive disease (PD) as determined by irRECIST, secondarily by mRECIST (HCC subjects only), intolerable toxicity or when the risk/benefit ratio is no longer beneficial for the subjects as determined by the Principal Investigator, or upon subject withdrawal of consent. Upon permanent discontinuation of study treatment, there is a Treatment Termination visit and a 30-Day Safety Follow-up visit. Subjects who drop out before they complete the first cycle of combination treatment for reasons other than toxicity are replaced.

Tolerability and safety of study treatment are evaluated throughout the study by collection of clinical and laboratory data, including information on adverse events (AEs), serious adverse events (SAEs), DLTs, concomitant medications, vital signs, electrocardiograms (ECGs), and Eastern Cooperative Oncology Group (ECOG) performance status. Antitumor response is assessed according to standard RECIST v1.1 and secondarily with irRECIST using computed tomography (CT) or magnetic resonance imaging (MRI) scans. Serum or plasma samples are collected for PK and PD analysis at specified time points.

Phase 1 and 2 assess the association of absolute neutrophil count (ANC) and neutrophil to lymphocyte ratio (NLR) at baseline and change in ANC and NLR ratio with combination treatment, to hepatocyte growth factor (HGF) and myeloid derived suppresser cells (MDSCs), and its correlation with pharmacokinetics.

The results indicate that the expression of HGF, the number of neutrophil and NLR correlate with the efficacy of the combination treatment.

1-26. (canceled)

27. A method of treating a subject having a cancer, the method comprising:
(A) administering to the subject a c-Met inhibitor which comprises a compound of the following formula 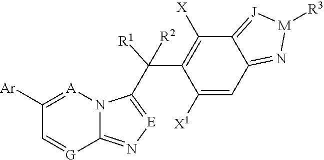
wherein:
R¹ and R² are independently hydrogen or halogen;
X and X¹ are independently hydrogen or halogen;
A and G are independently CH or N, or CH=G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH—C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl;
(B) administering to the subject an anti-PD-1 antibody or an anti-PD-L1 antibody.

28. The method of claim 27, wherein the c-Met inhibitor is selected from the group consisting of:

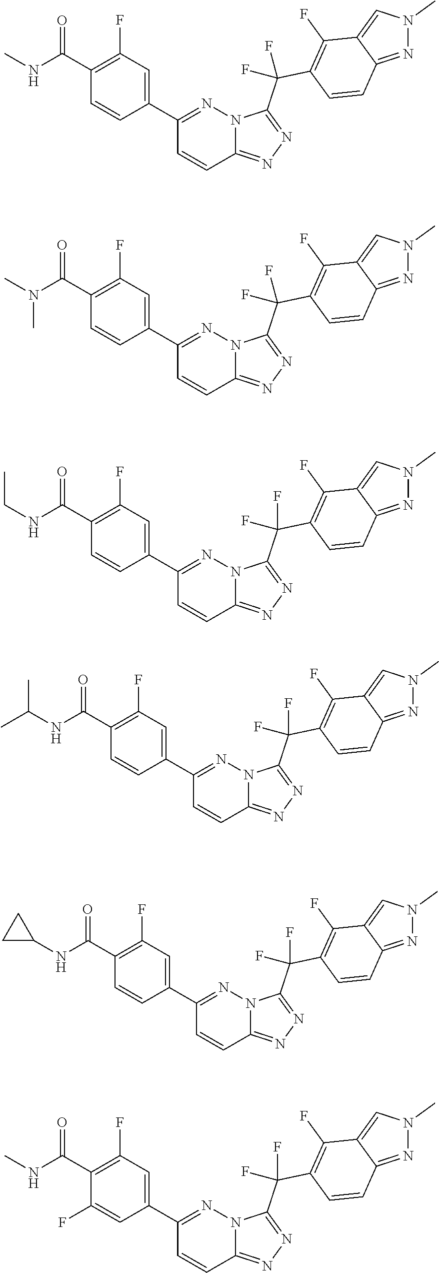

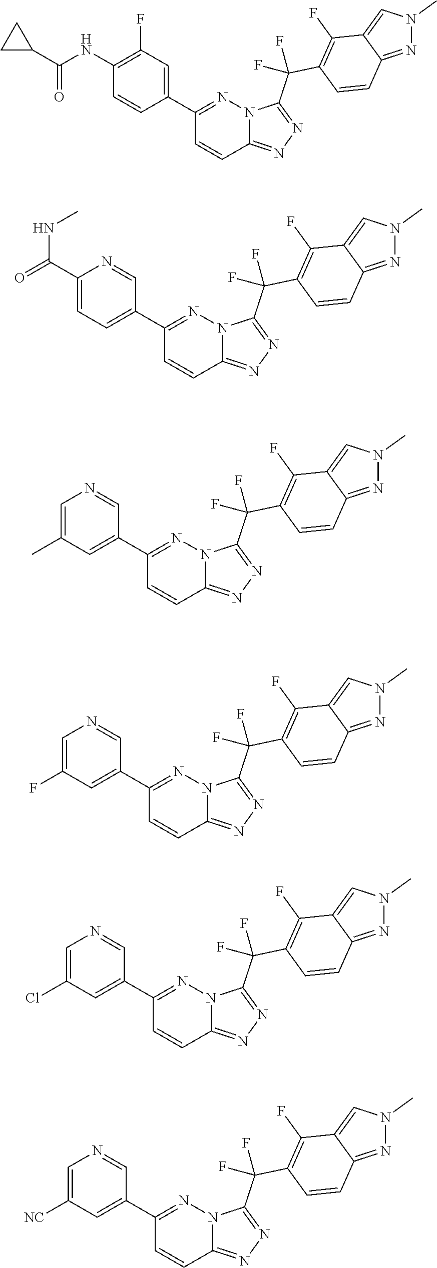

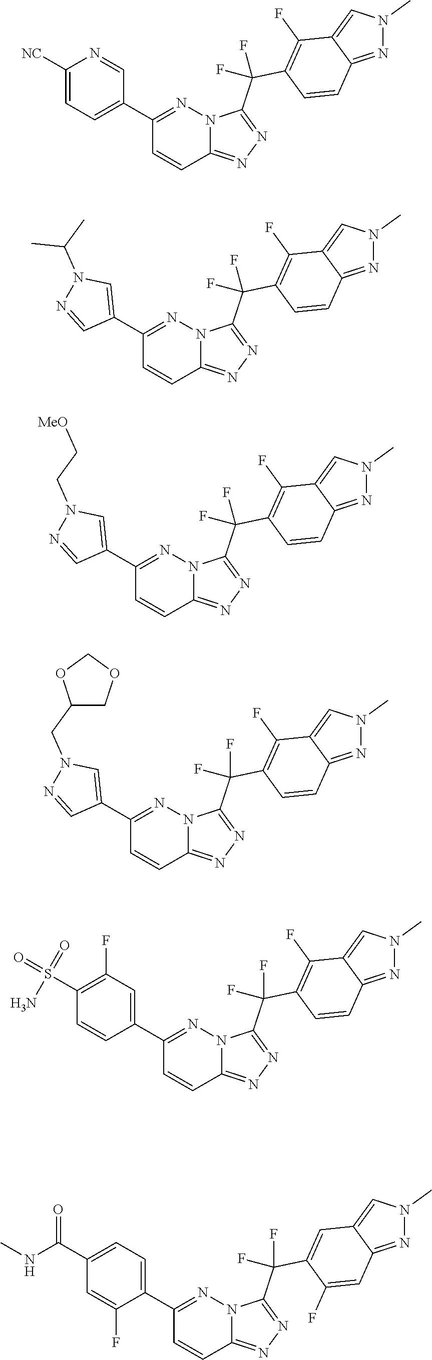

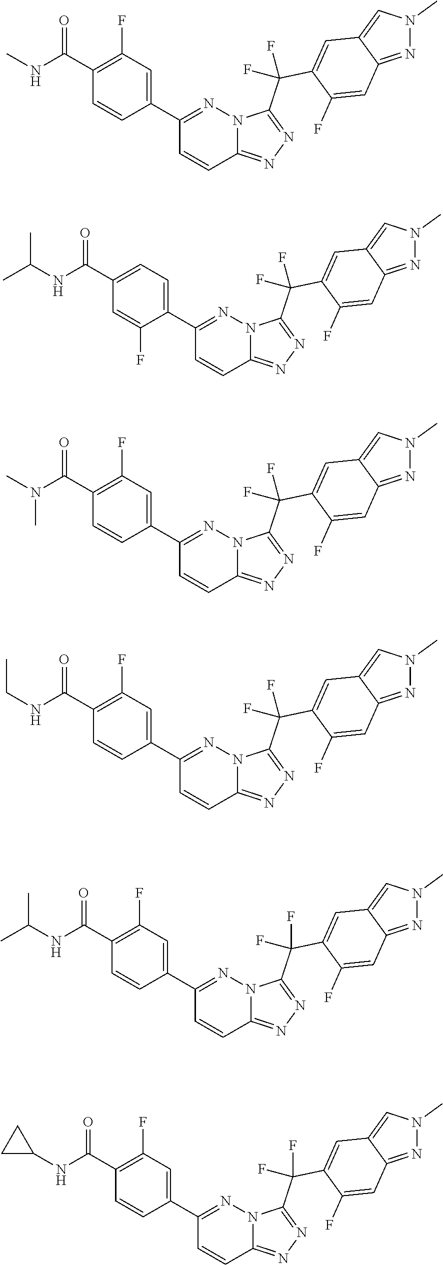

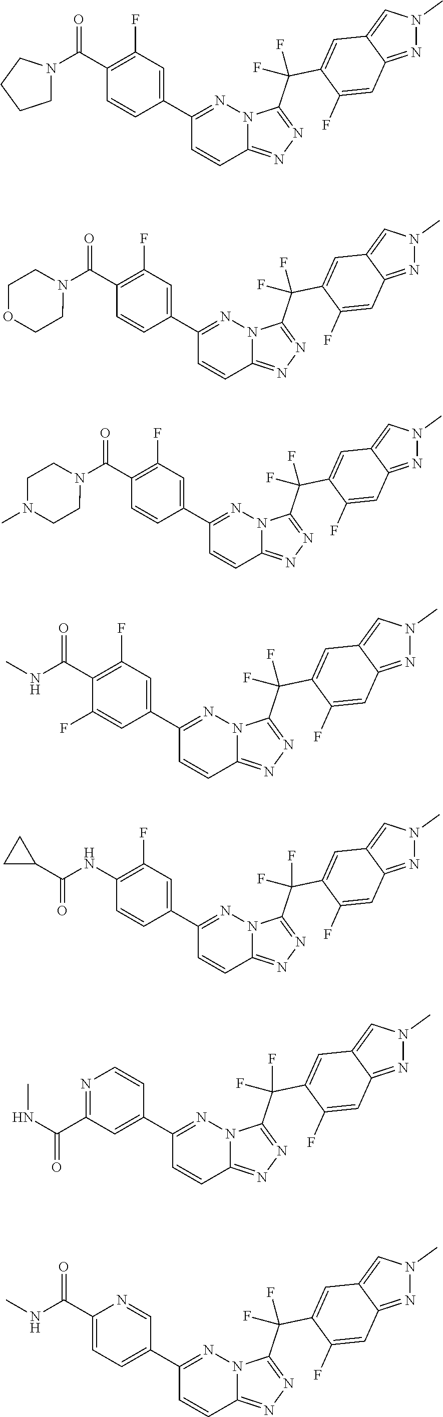

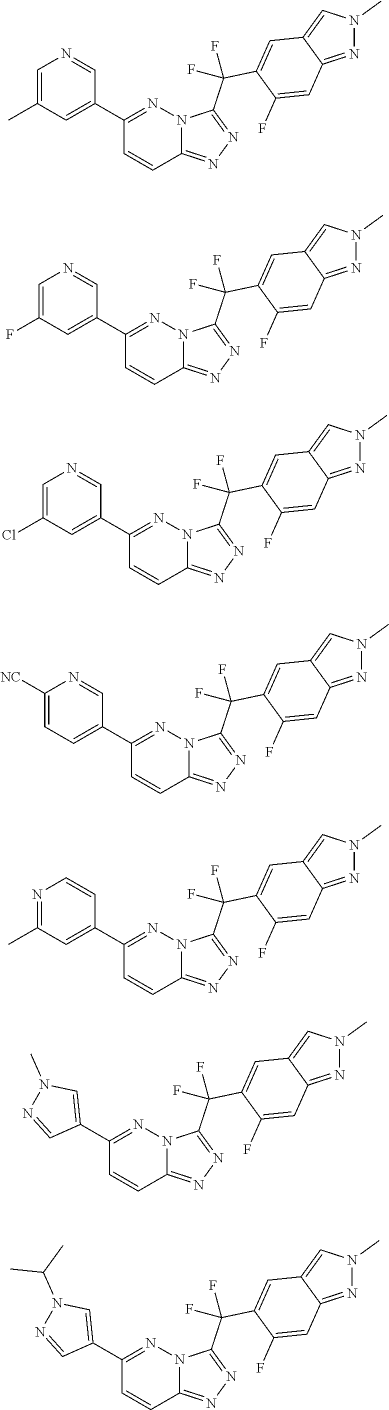

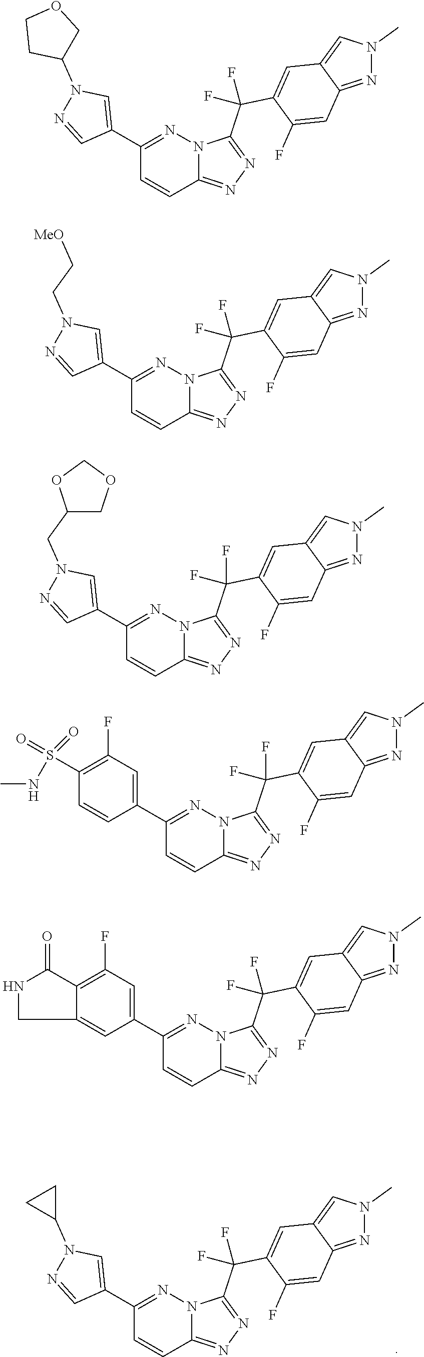

29. The method of claim 27, wherein the c-Met inhibitor is APL-101, which has the following formula:

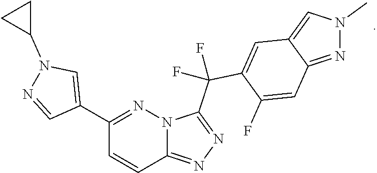 [SMILES: Cn1cc2cc(C(F)(F)c3nnc4ccc(-c5cnn(C6CC6)c5)nn34)c(F)cc2n1]

30. The method of claim 27, wherein the anti-PD-1 antibody is selected from the group consisting of those disclosed in WO2016/014688.

31. The method of claim 27, wherein the anti-PD-1 antibody is APL-501, GB226, or genolimzumab.

32. The method of claim 27, wherein the anti-PD-L1 antibody is selected from the group consisting of those disclosed in WO2016/022630.

33. The method of claim 27, wherein the anti-PD-L1 antibody is APL-502 or TQB2450.

34. The method of claim 27, wherein the cancer is selected from the groups consisting of a lung cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

35. The method of claim 27, wherein the cancer is a non-small cell lung cancer (NSCLC), renal cell carcinoma or hepatocellular carcinoma.
